(19) United States
(12) Patent Application Publication
(10) Pub. No.: US 2015/0320361 A1
(43) Pub. Date: Nov. 12, 2015

(54) METHOD FOR TESTING PLAUSIBILITY

(71) Applicant: SECA AG, Reinach BL 1 (CH)

(72) Inventors: Björn JENSEN, Hamburg (DE); Jan-Erik GROSSMANN, Hamburg (DE)

(21) Appl. No.: 14/655,410

(22) PCT Filed: Jan. 29, 2013
(86) PCT No.: PCT/DE2013/000060

§ 371 Date: Jun. 25, 2015

(51) Int. Cl. A61B 5/00; A61B 5/053

(52) U.S. Cl. CPC A61B 5/7221; A61B 5/053

(57) ABSTRACT

A method for testing the plausibility of electric impedance measurement values. The measurement values are determined during the measurement of the bio-impedance of a person. Real parts and imaginary parts of the impedance measurement values are determined for a plurality of different frequencies and are localized in a complex representation plane. The representation plane is defined by a coordinate axis for the imaginary part and a coordinate axis for the real part. The localization of the measurement values in the complex representation plane is compared to a desired profile, and the measurement values are adjudged to be not plausible if predefinable deviation from the desired profile is exceeded.

The invention pertains to a method for testing the plausibility of electrical impedance measurements acquired during a measurement of the bioimpedance of a person.

The measurement of electrical impedance of a person is typically done by the use of a so-called “body composition analyzer” (BCA) to obtain information on the composition of the body of the person in question. In the normal case, the percentages by weight of muscle mass, bone, fat, and water are determined. When these types of measurement methods are carried out, the person frequently is positioned on a measuring device, standing with each foot on one of a pair of electrodes. Hand electrodes are also gripped or contacted manually.

The quality of the measurement values in question and thus also the quality of the information derived from them concerning the composition of the body depend on many different interference factors. These are, for example, the body posture, the quality of the contact with the electrodes, and the positioning of the electrodes relative to the body.

In addition, there is often the problem that the person in question does not stand still during the performance of the measurement but rather moves around instead. This has the result that, when multiple measurement values are acquired in sequence, both accurate and inaccurate measurement values are obtained.

When measurements of the electrical impedance in question are to be carried out, the goal is to manage with the shortest possible measurement time and to take into account only qualitatively “good” measurement values. With the methods known so far, this cannot be done in a completely satisfactory manner.

The goal of the invention is to improve a method of the type described above in such a way that the measurements can be checked automatically for plausibility.

This goal is achieved according to the invention in that

the real parts and imaginary parts of the impedance measurement values are determined for a plurality of different frequencies and compared with respect to their localization in a complex representation plane defined by a coordinate axis for the imaginary part and a coordinate axis for the real part with a nominal curve, and in that

the measurement value is considered implausible if it exceeds a predefinable deviation from the nominal curve.

The method according to the invention can be visualized graphically by plotting a nominal curve, which shows a typical positioning of the real and imaginary parts for all frequencies between zero and infinity, in the complex plane. If the distance between the real part and/or the imaginary part of a concrete measurement value and the point on the nominal curve belonging to its measurement frequency exceeds an allowable maximum with respect to absolute value and/or phase, the measurement value in question is not plausible.

Not only is the geometric course of the nominal curve known, but each point on this curve can also be uniquely assigned to a measurement frequency. As a result, it is possible to determine, with respect to absolute value and phase, the deviation of a measurement value which has been obtained at a concrete measurement frequency from the associated point on the nominal curve.

With respect to the performance of the method according to the invention, what is envisioned in particular is not to use a nominal curve unchangeably localized in the complex number plane but rather to check, on the basis of the concretely determined measurement values, whether these values lie on a curve which corresponds in shape to the nominal one. During the performance of the method, therefore, it is possible, for example, to interpolate between measurement values and to determine the curve which thus results. This curve is then compared with respect to shape and frequency-dependent positioning of the individual measurement values with the originally defined nominal curve.

It is irrelevant in particular whether or not the curve specified by the determined measurement values is shifted, for example, versus an expected curve shape with respect to the real parts and/or the imaginary parts or whether a different scaling occurs. The essential point is that there be agreement with the predetermined curve shape.

In particular, it is envisioned that, during the performance of a measurement, a frequency-dependent series of measurements is conducted, which determines individual measurement values at different measurement frequencies and stores them. These measurement frequencies extend from very small values to very large values, as large as can be technically realized. For the later evaluation, only the measurement values found to be plausible are used.

A typical performance of the method is defined in that a section of a circle in the complex plane is used as the nominal curve.

According to a typical measurement procedure, it is provided that the frequency is changed from a minimum value to a maximum value, during the course of the measurement.

It is envisioned in particular that the minimum value is zero and the maximum value is infinity.

To ensure the most accurate possible use of the nominal curve, it is proposed that at least three measurement values be determined.

It has been found especially advantageous to determine at least eight measurement values.

Exemplary embodiments of the invention are illustrated schematically in the drawings:

FIG. 1 shows an equivalent electrical circuit diagram of the human body during the measurement of the bioimpedance;

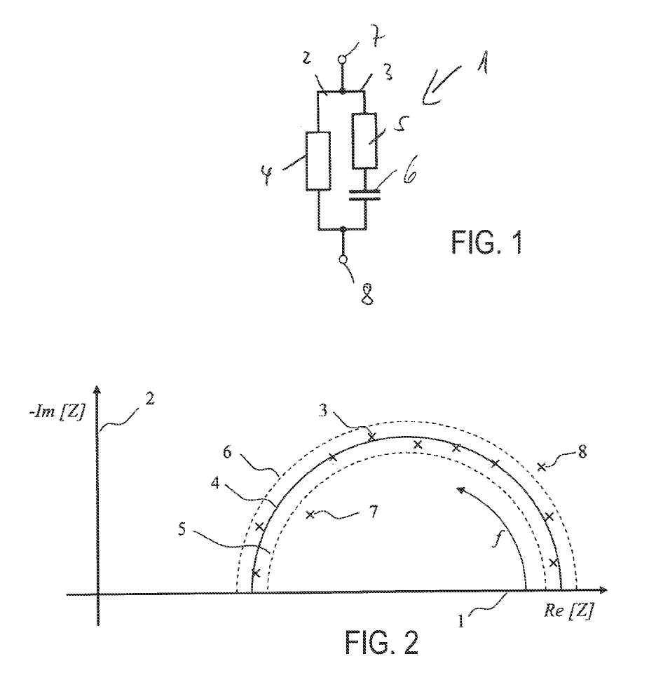

FIG. 2 shows a diagram of bioimpedance measurement values and a reference curve in the complex number plane;

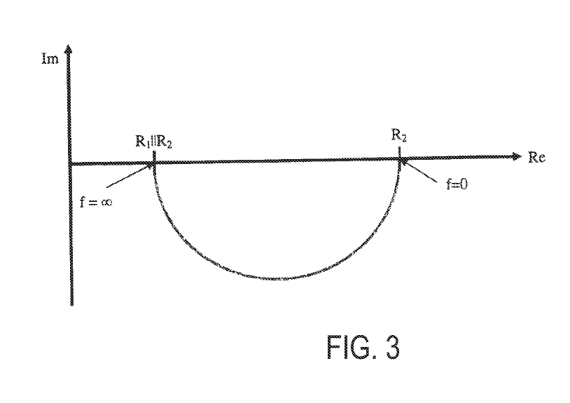

FIG. 3 shows a locus for the electrical resistance of the arrangement according to FIG. 1 at a frequency range from zero to infinity;

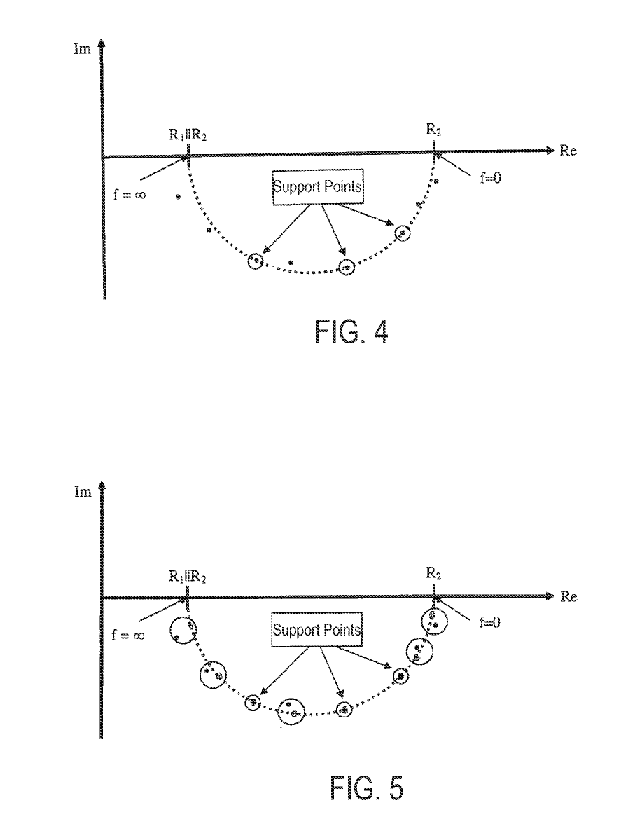

FIG. 4 shows a diagram illustrating the determination of a fitted curve by means of support points taken from a series of measurement values;

FIG. 5 shows a diagram illustrating a comparison of the existing measurement values with calculated values based on the fitted curve of FIG. 4;

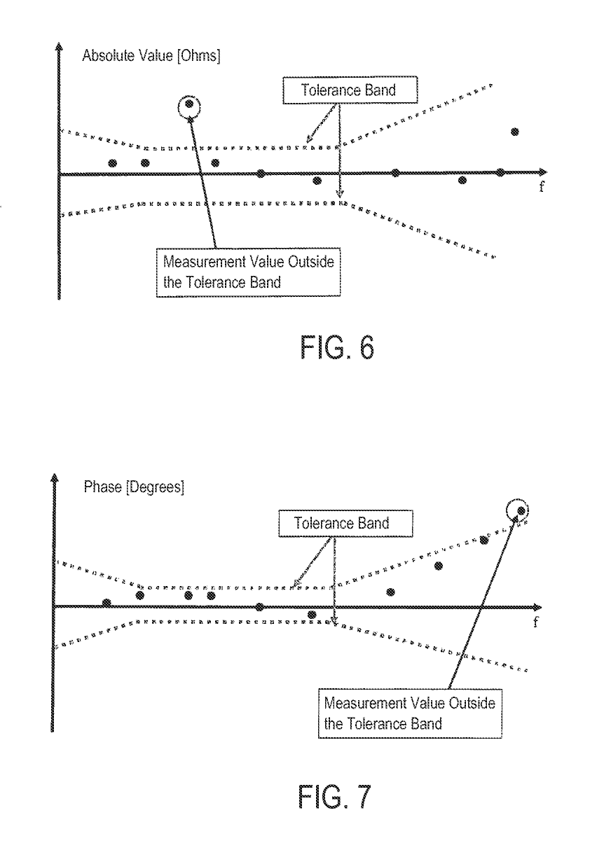

FIG. 6 shows a graph of the deviations of the absolute values of measurements of a measurement series from associated points on the fitted curve and a check of whether or not the measurement values are within a predefined tolerance band;

FIG. 7 shows another graph illustrating phase deviations of measurements of a measurement series from associated points on the fitted curve and the corresponding check of whether the measurements values are within a predefined tolerance band; and

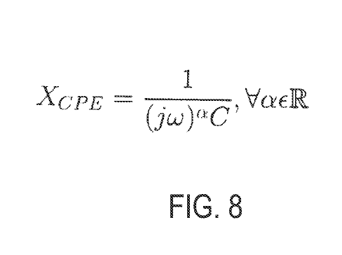

FIG. 8 shows a formula for calculating the complex impedance of a “constant phase element”.

FIG. 1 shows an equivalent electrical circuit diagram 1 for the human body during the measurement of the bioimpedance. The equivalent circuit diagram comprises two parallel branches 2, 3, wherein a resistor 4 is present in a branch 2, and a resistor 5 and a capacitor 6 are connected in series in the branch 3. The branches 2, 3 are brought together at the terminals 7, 8.

The capacitor 6 in the equivalent circuit diagram 1 is shown only as an example. In particular, it is also envisioned that, instead of the capacitor 6, a so-called “constant phase element” could be used.

When DC voltage is applied to the terminals 7, 8, only the resistor 4 is electrically active, because of the capacitor 6.

When AC voltage is applied to the terminals 7, 8, the resistors 4, 5 become connected progressively more in parallel with increasing frequency. Because of the complex impedance of the “constant phase element” 6, however, a phase shift is observed.

The constant-phase element behaves largely like a non-ideal capacitor.

FIG. 2 shows the impedance of the equivalent circuit diagram 2 within the complex number plane in the form of a locus 9. It can be seen that, when the DC current falls and thus for a frequency of zero, the impedance has only a real part, wherein this is determined by the resistor 4. As the frequency increases, the real part decreases, and the absolute value of the imaginary part increases at first. At an infinite frequency, the absolute value of the imaginary part of the impedance returns to zero again, and the impedance has only a real part, which results from the parallel connection of the resistors 4, 5.

The exact semi-circular locus 9 shown is obtained under consideration of the capacitor 6 in the equivalent circuit diagram 1. If, instead of the capacitor 1, the previously mentioned constant-phase element is used, then the locus 9 has the form of a shorter section of a circle.

The nominal curve can be determined geometrically, for example. The section of the circle in question is determined on the basis of three support points from the measurement series, and then the values of the components of the equivalent circuit diagram are determined. The intersection between the locus and the real axis at the greatest distance from the imaginary axis corresponds to the value of the resistor 4. The intersection between the locus and the real axis at the shortest distance to the imaginary axis corresponds to the value of the parallel connection of the two resistors 4, 5. The values of these two resistors are defined by this means.

The complex impedance of the constant-phase element is obtained from the formula of FIG. 8.

1-6. (canceled)

7. A method for testing plausibility of electrical impedance measurements obtained during a measurement of bioimpedance of a person, the method comprising the steps of:
determining real parts and imaginary parts of the impedance measurements for a plurality of different frequencies and comparing the real and imaginary parts with respect to their localization in a complex representation plane defined by a coordinate axis for the imaginary part and a coordinate axis for the real part with a nominal curve; and considering a measurement value as not plausible if the measurement value exceeds a predefinable deviation from the nominal curve.

8. The method according to claim 7, including using a semi-circle in the complex plane as the nominal curve.

9. The method according to claim 7, including changing the frequency from a minimum value to a maximum value during performance of the measurement.

10. The method according to claim 9, wherein the minimum value is 0 Hz and the maximum value is infinity.

11. The method according to claim 7, wherein at least three measurement values are determined.

12. The method according to claim 11, wherein at least eight measurement values are determined.
